(19) United States
(12) Patent Application Publication
(10) Pub. No.: US 2013/0171630 A1
(43) Pub. Date: Jul. 4, 2013

(54) METHODS OF USING TELOMERES AS MARKERS FOR AGING

(71) Applicant: James QIN, Clarksville, MD (US)

(72) Inventor: James QIN, Clarksville, MD (US)

(73) Assignee: QIAGEN Inc., Germantown, MD (US)

(21) Appl. No.: 13/445,097

(22) Filed: Apr. 12, 2012

Related U.S. Application Data
(60) Provisional application No. 61/581,328, filed on Dec. 29, 2011.

(51) Int. Cl. G01N 21/64; C12Q 1/68

(52) U.S. Cl. 435/6.11

(57) ABSTRACT

The invention relates to a simple, reproducible, fast, and accurate method of quantifying and measuring telomeres in a clinical sample. The invention further relates to kits comprising premixed and optimized buffers, DNA polymerase, primers, and instructions for the detection of telomere length and quantities. Also envisioned are complete kits further including instrumentalities for the detection of telomere length and quantities.

### PRIORITY

This application is a Utility of U.S. Provisional Application No. 61/581,328, filed Dec. 29, 2011, the disclosure of which is hereby incorporated by reference in its entirety.

### FIELD OF INVENTION

The invention generally relates to a simple, reproducible, fast, and precise method of quantifying and measuring telomeres in clinical samples. In one aspect, the invention relates to methods of measuring the length of telomeres to determine aging by using the primers and probes of the invention in amplification reactions. Specifically, the amplification reactions utilized by the present invention include quantitative real time polymerase chain reaction (“qPCR”). In another aspect of the invention, the invention relates to methods of detecting the abundance of telomere hexameric repeating units and determining the length and frequency of telomere repeat sequences for the diagnosis of a diseases or conditions such as age related telomere disease selected from bone marrow failure, leukemia, macular degeneration, atherosclerosis, impaired wound healing, heart disease, wrinkling, or age related graying of hair.

### BACKGROUND

Telomeres are the ends of linear chromosomes and are composed of tandem hexameric nucleotide repeats of 5′-TTAGGG-3′. The main function of telomeres is to protect the natural ends of chromosomes from being recognized as damaged DNA. Due to DNA polymerase's inability to fully duplicate the DNA strands at the chromosomal extremities, telomeres shorten with each cell division thereby resulting in the shortening of telomeres with age. Over time, the lose of telomeres contributes to chromosomal instability.

To maintain telomeres, cells with highly proliferative capacities express telomerases. Telomerases are reverse transcriptase enzymes that use a RNA template to elongate the 3′ end of the leading strand of telomeres to maintain their lengths. Some genetic diseases are caused by deficient telomerase function in which mutations in the telomerase complex are etiologic. In these diseases, telomeres are extremely short. Typically, patients will manifest clinical symptoms of cell senescence and chromosomal instability. Some patients with telomerase mutations will also present with bone marrow failure and an increased propensity for the development of leukemia.

Precise, reproducible, and simple methods aimed at measuring telomere length are highly desired in both the laboratory and in the clinical practice. Currently three major methods are currently available in the laboratory to measure telomere length. These are Southern blotting, flow-fluorescent in situ hybridization (“flow-FISH”), and qPCR. Southern blotting, the gold standard method of measuring telomere length, is generally recognized as being laborious, time consuming, and requiring large quantities of DNA for the analysis. Flow-FISH, a technique which combines flow cytometry and fluorescent in situ hybridization, is also viewed as laborious and requiring intact cells for analysis. Finally, qPCR requires low quantities of DNA, but the method requires several reagents to stabilize the reaction into a “homemade” master mix.

Others have developed methods for the measurement of telomeres by using qPCR. For example, Cawthon et al (Nucleic Acid Research, 2002, vol. 30(10): e47) developed a method of measuring telomere length by using primers that bind to TTAGGG and CCCTAA hexameric repeats present in telomeres. Because primers that bind to the telomere hexameric repeats have a tendency to form primer dimers, Cawthon developed primers that comprised specifically placed mismatched nucleotide bases. When compared to Southern blotting, Cawthon's qPCR measurement correlated to approximately 67% (R²=0.6771 by measuring telomere to single copy gene ratios), suggesting that the method is a possible replacement for the Southern blotting technique. Certainly, qPCR can be used as a method of measuring telomere length, with some degree of accuracy and correlation to Southern blotting. However, the method described in the prior art is extremely time consuming and laborious.

Thus, there is a need to have a simple, accurate, and reproducible method for (i) determining the abundance of telomeres hexameric repeats and (ii) determining telomere length. The present invention describes a simplified and automated qPCR method for measuring telomere length using standardized, commercially available chemistry, which is highly reproducible and accurate for peripheral cells.

### SUMMARY

The present invention provides a method of quantitatively measuring the nucleic acid sequence length of a telomere DNA sequence, and/or determining the abundance of telomeric hexameric repeats.

It is an object of the present invention that the measurement of the telomere nucleic acid sequence is a means of detecting chromosomal instability, a means for determining aging; a means for diagnosis diseases and/or conditions relating to or associated with age related telomere disease or condition selected from bone marrow failure, leukemia, macular degeneration, atherosclerosis, impaired wound healing, heart disease, wrinkling, or age related graying of hair.

It is yet another object of the present invention that the measurement of the telomere nucleic acid sequence is accomplished by amplification of tandem hexameric sequences present in telomere region of chromosomes. The hexameric sequence is characterized by tandem repeating sequences of 5′-TTAGGG-3′.

It is still another object of the present invention that the amplification of the telomere region is accomplished by polymerase chain reaction (PCR). More specifically, it is another embodiment of the present invention that the amplification of the telomere region is accomplished by real time qPCR.

It is yet another object of the present invention that the nucleic acid templates used for the amplification of the telomere region are derived from the tissue samples. More specifically, the genomic DNA samples are derived from any peripheral cells, such leukocytes, primary and secondary cell lines, cancer cell lines, cancer cells, cells isolated from eukaryotic tissues and extracted from any tissues. The extraction of the sample may be performed manually or automated.

It is still another object of the present invention that the amounts of starting nucleic acid DNA template used for the amplification reaction is significantly decreased. The decreased amounts of starting template DNA increases the sensitivity of the amplification. Unexpectedly, an increase in the accuracy of the amplification can be achieved for the tandem hexamer of the telomere region by lowering the concentration of starting template DNA. The template DNA can range from about 0.1 nanograms (ng) to about 20 ng. More specifically, between 0.1 ng to 6.25 ng.

It is a further object of the present invention that the amplification reaction occurs in the presence of a reaction mixture that has been optimized for the amplification of the telomere region. Such optimizations may include the concentrations of MgCl₂, NH₄⁺, and/or K⁺; the available units of Taq polymerase enzyme; the concentrations of the forward and reverse primers; the addition of specific additives that alter the melting behavior of nucleic acid templates, such as betaine (e.g., solution Q) and/or additives that enhance multiplex or primer annealing behaviors (e.g., synthetic factor MP).

It is still another object of the present invention that the amplification reaction occurs for a duration of time that is significantly abbreviated when compared to known amplifications reactions taught in the prior art. More specifically, the amplification times of the telomere hexameric DNA sequence can be accomplished within one hour (as compared to one hour and 45 minutes) for 25 cycles of amplification. Control single gene copy reactions can be accomplished in approximately one hour (as compared to two hours and 20 minutes) for 35 cycles of amplification. Thus this decrease in amplification time provides an improvement in the ability to diagnosis certain diseases and conditions associated with telomere lengths and abundance of repeating hexameric units.

It is a further object of the present invention that the amplification reaction occurs at a temperature that is above the standard denaturation temperature for double stranded DNA (i.e., standard temperature range of about 93° C. to about 95° C.) and for a duration in the range of about 5 seconds to about 30 seconds. Additionally, the amplification reaction also occurs at a temperature range above the normal annealing temperature for primers (i.e., normal annealing temperature of about 58° C.) for a duration in the range of about 5 seconds to about 30 seconds.

It is yet another object of the present invention to perform the amplification reaction on automated instruments. The automated instruments may include thermocyclers, real time thermocyclers and may include, but are not limited to, QIAGEN RotorGene, ABI 7000, ABI7500, ABI 7900HT, Roche LightCycler 1.2, Roche LightCycler 2.0, Roche LightCycler 480, MJ Research Chrom4 Cycler, PTC-100, PTC-200, PTC-225, PTC-240, and Stratagene Robocycler Gradient 96 Gradient Thermal Cycler.

It is an additional object of the present invention to analyze or detect the presence of the amplified product by detecting an amplified labeled product. This may include, amongst others, dyes or markers that are capable of binding to double stranded DNA.

It is yet another object of the present invention to diagnose or prognosticate on the presence of a particular disease or condition associated with the shortening of the telomere sequence. These diseases may include, for example, macular degeneration (vision loss), atherosclerosis (hardening of arteries by plaques), impaired wound healing, heart disease, gray hair and/or wrinkles.

The present invention is also envisioned to be useful as a single all-inclusive kit that includes the appropriate buffers, primers, amplification enzymes, instruction pamphlets that provide for optimal temperatures, cycles, and conditions for the rapid detection of a target repeating hexameric telomere sequences. This kit may further comprise a machine which is capable of performing the amplification of the telomere sequence.

### BRIEF DESCRIPTION OF THE FIGURES

FIG. 1: Telomere assay for the determination of standard curve.

FIG. 2: Telomere assay with diluted telomere gDNA from peripheral cells.

FIG. 3. Single Gene (Reference Gene) Assay.

FIG. 4. Single Gene (Reference Gene) with dilute gDNA from peripheral cells.

FIG. 5. Telomere length correlation between Southern blotting and qPCR.

FIG. 6. Telomere length of healthy individuals as a function of age.

### DETAILED DESCRIPTION OF THE PREFERRED EMBODIMENTS OF THE INVENTION

It has been found in accordance to the present invention that the amplification of a tandem hexameric repeating sequence in a telomere gene has the ability to help diagnose the presence of chromosomal instability related diseases and/or conditions. It has particularly been found by the present invention that by altering the reaction conditions and reagents, the detection of the hexameric regions of the telomere can be accurately, precisely, and quickly assessed from limited quantities of starting DNA materials. Surprisingly, by altering various parameters unknown or unexpected by those of skill in the art, the present invention is capable of improving upon the known quantitative methods of measuring telomere length and abundance of hexameric repeating units.

A. DNA Templates

As with most nucleic acid amplification methodologies, the starting point is the extraction of the nucleic acid molecules from a sample. The sample comprising the target sequence may be obtained from any tissue of any organism, including blood, brain, bone marrow, lymph, liver, spleen, breast, epithelia (e.g., skin, mouth, etc.), or other tissues, including those obtained from biopsy. The samples may also comprise bodily excretions or fluids, such as saliva, urine, feces, cerebrospinal fluid, semen, milk, etc. Other sources of target nucleic acids include bacteria, yeast, plant, virus, or other nucleic acid containing organisms, pathogenic or nonpathogenic. The nucleic acid can also be any nucleic acid generated artificially by chemical or enzymatic processes, such as PCR reactions. In one embodiment of the present invention, the source of the target sequence is derived from peripheral cells, such as blood cells.

DNA may be isolated from the sample using conventional methods. One example of a method of removing DNA is a technique employing the isolation of long DNA fragments, such as the use of agarose plugs described by Heiskanen et al., Biotechniques 17, 5, 928-929; 9320933, 1994.

Those of skill in the art are generally aware and accustom to the various methods of extracting DNA and RNA nucleic acids molecules. These methods may include treating using detergents, sonication, electroporation, denaturants, etc., to disrupt the cells, bacteria, or viruses. The target nucleic acids may be purified as needed. The samples may also be extracted using kits or automated procedures or instruments. The automated instruments may include, but not limited to, QIAcube, QIAsymphony, EZ1 advanced, Biorobot Universal, QIAextractor, or QIAgility.

By “target nucleic acid,” “target sequence,” “target DNA,” “template DNA,” or grammatical equivalents herein is meant a nucleic acid sequence on a double or single stranded nucleic acid. The target sequence may be a portion of a gene, genomic DNA (“gDNA”), cDNA, RNA, including mRNA and rRNA, or other nucleic acids. In one preferred embodiment, the target is gDNA comprising a telomere sequence derived from any species.

As will be appreciated by those skilled in the art, the target sequence may take many forms. For example it may be contained within a larger nucleic acid sequence, i.e., all or part of a gene or mRNA, a restriction fragment of a plasmid or genomic DNA, among others.

The target sequence or template may be any length, with the understanding that longer sequences are more specific. In some embodiments, it may be desirable to fragment or cleave the sample nucleic acid into fragments of 100-10,000 base pairs, with fragments of roughly 500 base pairs being preferred in some embodiments. Fragmentation or cleavage may be done in any number of ways well known to those skilled in the art, including mechanical, chemical, and enzymatic methods. Thus, the nucleic acids may be subjected to sonication, French press, shearing, or treated with nucleases, or chemical cleavage agents.

B. Reaction Mixtures

A typical reaction mixtures for the present invention may include a polymerase, free nucleotide bases (e.g., dNTPs), MgCl₂, buffers, additives, template or target DNA, and forward and reverse primers.

Those of skill in the art will appreciate that the type of polymerases used for the amplification of target sequences may be varied according to the specific reaction conditions desired. Preferred polymerases are thermostable polymerases lacking 3′ to 5′ exonuclease activity since use of polymerases with strong 3′ to 5′ exonuclease activity tends to remove the mismatched 3′ terminal nucleotides. Accordingly, a variety of polymerases, which are commercially available from a variety of sources, may be used in the present invention. These include, but are not limited to, Taq DNA polymerases (e.g., TopTaq DNA polymerase), DNA proofreading polymerases (e.g., Pfu DNA polymerases and Vent_{R} DNA polymerases), polymerases designed for long extensions or high fidelity polymerases (e.g., LongAmp Taq, HotStar Hifidelity polymerases), hot start polymerases (e.g., HotStar and HotStar plus DNA polymerases), etc. Also useful are polymerases engineered to have reduced or non-functional 3′ to 5′ exonuclease activities (e.g., Pfu(exo-), Vent(exo-), Pyra(exo-), etc. Also applicable are mixtures of polymerases used to optimally extend hybridized primers.

In another aspect, polymerase enzymes useful for the present invention are formulated to become active only at temperatures suitable for amplification. For example, the enzymes are in an inactive form rendering it unavailable until a specific amplification temperatures is reached. These polymerase formulations allow mixing all components in a single reaction vessel while preventing priming of non-target nucleic acid sequences. In one preferred embodiment, the polymerase used in the present invention is a “hot start” enzyme that is activated at a temperature of about 95° C.

In another aspect, the person skilled in the art can use various nucleotide analogs for amplification of particular types of sequences, for example GC rich or repeating sequences. These analogs may include, among others, c⁷-dGTP, hydroxymethyl-dUTP, dITP, 7-deaza-dGTP, etc.

In another aspect, those skilled in the art will appreciate that various agents may be added to the reaction to increase the productivity of the polymerases, stabilize the polymerases from inactivation, decrease non-specific hybridization of the primers, or increase efficiency of replication. Such additives include, but are not limited to, dimethyl sulfoxide (“DMSO”), formamide, acetamide, glycerol, polyethylene glycol, or proteinacious agents such as E. coli. single stranded DNA binding protein, T4 gene 32 protein, bovine serum albumin, gelatin, etc. In one embodiment of the invention, the reaction mixture does not include any additives such as DMSO to improve the reaction. In another embodiment, the concentration of MgCl₂ need not be altered between the target DNA and the single gene reference.

A variety of agents may be added to the reaction to facilitate optimal hybridization, amplification, and detection. These include salts, buffers, neutral proteins, detergents etc. Other agents may be added to improve efficiency of the reaction, such as protease inhibitors, nuclease inhibitors, anti-microbial agents, etc., depending on the sample preparation methods and purity of the target nucleic acid. Components of the reaction may be added simultaneously, or sequentially, in any order as outlined below. In one embodiment of the invention, the reaction mixture may comprise an agent that affects the melting behavior of nucleic acid templates, such as betaine (e.g., solution Q) and/or additives that enhance multiplex or primer annealing behaviors (e.g., synthetic factor MP).

As indicated above, a variety of template sequences may be used in the present invention. In one embodiment of the invention, the target sequence comprises a human telomere sequence. As the present invention surprisingly discovered, lowering the amount of starting nucleic acid template produced an unexpected increase in the sensitivity of detecting telomere length and hexameric repeating units by qPCR. The prior art teaches that the measurement of telomere length typically required an amount of approximately 35 ng per reaction of telomere DNA. However, the present invention, lowered the amount to be in a range from about 0.1 ng to about 20 ng. By doing so, there was an unexpected increase in the ability to accurately measure the length of telomere sequences. In one embodiment of the invention, the lowered amount of telomere DNA is in the range of about 0.1 ng to about 10 ng, preferably in the range of about 0.1 ng to about 5 ng, more preferably, in the range of about 0.1 ng to about 1 ng. In one embodiment the amount of telomere DNA is about 0.1 ng, 0.2 ng, 0.3 ng, 0.4 ng, 0.5 ng, 0.6 ng, 0.7 ng, 0.8 ng, 0.9 ng, 1.0 ng, 1.5 ng, 2.0 ng, 2.5 ng, 3.0 ng, 3.5 ng, 4.0 ng, 4.5 ng, 5.0 ng, 5.5 ng, 6.0 ng, 6.5 ng, 7.0 ng, 7.5 ng, 8.0 ng, 8.5 ng, 9.0 ng, 9.5 ng, 10.0 ng, 10.5 ng, 11.0 ng, 11.5 ng, 12.0 ng, 12.5 ng, 13.0 ng, 13.5 ng, 14.0 ng, 14.5 ng, 15.0 ng, 15.5 ng, 16.0 ng, 16.5 ng, 17.0 ng, 17.5 ng, 18.0 ng, 18.5 ng, 19.0 ng, 19.5 ng, 20.0 ng, or any nanogram interval amount therein.

Generally, the method of the present invention provide for a first primer which hybridizes to a first single strand of the target nucleic acid and a second primer which hybridizes to a second single strand of the target nucleic acid, where the first and second strands are substantially complementary. The primers are capable of primer extension by polymerase when hybridized to their respective strands. That is, the primers hybridized to the target nucleic acid have their 3′ terminal nucleotide residues complementary to the nucleotide residue on the target nucleic acid such that the primers are capable of primer extension. Accordingly, “primers,” “probes,” “nucleic acid primers,” “oligonucleotide primers,” “primer nucleic acid”, or grammatical equivalents thereof as used herein is meant a nucleic acid that will hybridize to some portion of the target nucleic acid. The primers of the present invention are designed to be substantially complementary to a target sequence such that hybridization of the target sequence and the primers of the present invention occurs, and proper 3′ base pairing allows primer extension to take place.

The term “complementary” or “substantially complementary” herein is meant that the probes are sufficiently complementary to the target sequences to hybridize under normal reaction conditions. Deviations from perfect complementary are permissible so long as deviations are not sufficient to completely preclude hybridization. However, if the number of alterations or mutations is sufficient such that no hybridization can occur under the least stringent of hybridization conditions, as defined below, the sequence is not a complementary target sequence. As such complementarity need not be perfect. In one embodiment of the present invention, the complementarity between the primers and the target telomere sequence is completely complementary such that there is no deviation. In one embodiment, the forward primer for the amplification of the telomere DNA comprises SEQ ID No.: 1 and the reverse primer for the amplification of the telomere DNA comprises SEQ ID No.: 2. In one embodiment the forward primer for the amplification of the telomere DNA is SEQ ID No.: 1 and the reverse primer for the amplification of the telomere DNA is SEQ ID No.: 2.

The size of the primer nucleic acid may vary, as will be appreciated by those in the art, in general varying from 5 to 500 nucleotides in length, with primers of between 10 and 100 nucleotides being preferred, between 12 and 75 nucleotides being particularly preferred, and from 15 to 50 nucleotides being especially preferred, depending on the use, required specificity, and the amplification technique. In one embodiment, the primer used in the present invention is approximately 39 nucleotides long.

In one preferred embodiment, the method for amplifying hexameric units of a repetitive region of a telomere sequence comprises a first primer which hybridizes to at least one repetitive unit or a region proximate thereto, on a first single strand of the target nucleic acid and a second primer which hybridizes to at least one repetitive unit or a region proximate thereto, on a second single strand of the target nucleic acid, where the first and second strands are substantially complementary. The primers are capable of primer extension when hybridized to their respective strands of the target nucleic acid.

In yet another preferred embodiment for amplifying repetitive hexameric telomere repeating regions, the present invention comprises a first primer which hybridizes to more than one repetitive unit or a region proximate thereto, on a first single strand of a target nucleic acid and a second primer which hybridizes to more than one repetitive unit or a region proximate thereto on a second single strand of the target nucleic acid, where the first and second strands are substantially complementary. The primers are capable of primer extension when hybridized to their respective strands of the target nucleic acid, as described above.

C. Amplification Reaction

Amplification reactions are generally carried out according to procedures well known in the art (see e.g., U.S. Pat. Nos. 4,683,195 and 4,683,202; hereby incorporated by reference). In brief, a double stranded target nucleic acid is denatured, generally by incubating at a temperature sufficient to denature the strands, and then incubated in the presence of excess primers, which hybridizes (i.e., anneals) to the single-stranded target nucleic acids. A DNA polymerase extends the hybridized primer, generating a new copy of the target nucleic acid. The resulting duplex is denatured and the hybridization and extension steps are repeated. By reiterating the steps of denaturation, annealing, and extension in the presence of a second primer for the complementary target strand, the target nucleic acid encompassed by the two primers is exponentially amplified. The time and temperature of the primer extension step will depend on the polymerase, length of target nucleic acid being amplified, and primer sequence employed for the amplification. The number of reiterative steps required to sufficiently amplify the target nucleic acid will depend on the efficiency of amplification for each cycle and the starting copy number of the target nucleic acid. Thus, in one preferred embodiment, the number of reiterative steps range from about 25 to 50 cycles, more preferably 25 to 40 cycles, even more preferably, 25 to 30 cycles. In another embodiment, the number of cycles for the amplification reaction is 25, 26, 27, 28, 29, 30, 31, 32, 33, 34, or 35 cycles.

The present invention relates to amplifying target nucleic acids with the primers described above. Thus in a preferred embodiment, the method comprises contacting a target telomere nucleic acid comprising a hexameric repeat sequence with a substantially complementary first and second strands with the first and second primers described above; and amplifying the target nucleic acid by polymerase chain reaction. Thus, in one embodiment of the present invention, the amplification target comprises a telomere hexameric repeat sequence. In yet another embodiment, the telomere hexameric repeat sequence is amplified using forward and reverse primers of SEQ ID Nos.: 1 and 2, respectively.

The conditions, such as enzyme concentrations, buffer systems, temperature and time of incubation require careful selection and depend upon factors such as the particular enzyme being used. In the present invention, it has been unexpectedly determined that the highly G/C rich hexameric telomere repeat sequences can be quickly and accurately amplified if certain conditions are modified from the standard amplification reactions.

The Taq Polymerase used in this invention is HotStarTaq Plus DNA Polymerase. It is a modified form of a recombinant 94 KDa DNA polymerase, originally isolated from Thermus aquaticus. This HotStar Taq plus DNA Polymerase is provided in an inactive state and has no enzymatic activity at ambient temperature. Thus, in one embodiment of the present invention, the initial heating step requires activation of the polymerase enzyme from an inactive state at a particular temperature and duration of time. Thus in one embodiment of the invention, the inactive enzyme is activated at a temperature of approximately 95° C. or exactly 95° C. for a duration in the range of approximately 2 minutes to 5 minutes. Those of skill in the art will be capable of determining this initial activation temperature based on the particular polymerase used in the amplification reaction.

Following enzyme activation, the double stranded DNA is denatured to separate the double stranded DNA. Typically, the DNA is heated to a temperature ranging from approximately 93° C. to 95° C. for a duration of approximately 5 to 30 seconds. Others who have amplified and measured telomere hexameric repeat lengths have used a denaturing temperature of 95° C. for 15 seconds (see e.g., Cawthon et al. at pg. e47-52) In the present invention, it was unexpected discovered that by raising the temperature to 98° C. for a duration of 5 to 10 seconds, there is an improved accuracy and amplification of the target hexameric telomere repeat sequence. Thus in one preferred embodiment of the invention, the target telomere sequence is denatured at a temperature of 98° C. for a period of 5 to 10 seconds.

After the double stranded DNA is denatured, the target sequence undergoes annealing and extension. The typical temperature for this stage of the amplification reaction is about 58° C. for a duration of approximately 10 to 60 seconds. In the present invention, however, it was unexpectedly discovered that by raising the temperature to 60° C., the degree of accuracy of the amplification reaction was increased. Thus in one preferred embodiment of the invention, annealing and extension phase of the amplification reaction is at a temperature of 60° C. for a duration of approximately 10 to 30 seconds.

The term “amplification” is meant to refer to a number of different methods of amplifying nucleic acid sequences. These include the polymerase chain reaction (PCR), qPCR, ligase chain reaction (LCR), self-sustained sequence replication (3SR), nucleic acid sequence based amplification (NASBA), strand displacement amplification (SDA), and amplification with Qβ replicase. In one preferred embodiment of the present invention, amplification refers to PCR or qPCR.

The products of the amplification are detected and analyzed by methods well known in the art. Amplified products may be analyzed following separation and/or purification of the products, or by direct measurement of product formed in the amplification reaction. Separation and purification methods include, among others, electrophoresis, including capillary electrophoresis (e.g., in agarose or acrylamide gels); chromatography (e.g., affinity, molecular sieve, reverse phase, etc.); and hybridization. The purified products may be subjected to further amplifications as is well known in the art. For detection, the product may be identified indirectly with fluorescent compounds, for example with ethidium bromide or SYBR™Green, or by hybridization with labeled nucleic acid probes. Alternatively, labeled primers or labeled nucleotides are used in the amplification reaction to label the amplification product. The label comprises any detectable moiety, including fluorescent labels, radioactive labels, electronic labels, and indirect labels such as biotin or digoxigenin. When indirect labels are used, a secondary binding agent that binds the indirect label is used to detect the presence of the amplification product. These secondary binding agents may comprise antibodies, haptens, or other binding partners (e.g., avidin) that bind to the indirect labels. Secondary binding agents are preferably labeled with fluorescent moieties, radioactive moieties, enzymes, etc.

In another preferred embodiment, the amplification product may be detected and quantitated during the amplification reaction by qPCR, or variations of which are well known in the art. For instance, a probe primer hybridizes to a sequence within the target nucleic acid, wherein the probe is labeled with two different fluorescent dyes (i.e., dual-labeled fluorogenic oligonucleotide probe), the 5′ terminus reporter dye and the 3′ terminus fluorescence quenching dye. Cleavage of the probe by the 5′ to 3′ exonuclease activity of DNA polymerase during the extension phase of PCR releases the fluorogenic molecule from proximity of the quencher, thus resulting in increased fluorescence intensity.

In another aspect, qPCR may be based on fluorescence resonance energy transfer (FRET) between hybridization probes (Wittwer, C. T. Biotechniques 22: 130-138 (1997); incorporated by reference in its entirety). In this method, two oligonucleotide probes hybridize to adjacent regions of the target nucleic acid sequence. The upstream probe is labeled at the 3′ terminus with an excitor dye (e.g., FITC) while the adjacently hybridizing downstream probe is labeled at the 5′ terminus with a reporter dye. Hybridization of the two probes to the amplified target nucleic acid sequences positions the two dyes in close spatial proximity sufficient for FRET to occur. This allows monitoring the quantity of amplified product during the polymerase chain reaction. A similar approach is used in the molecular beacon probes (Tyagi, S, Nat. Biotechnol. 16: 49-53 (1998); incorporated by reference). Molecular beacons are oligonucleotide probes comprising a quencher dye and a reporter dye at the opposite ends of a PCR product specific oligonucleotide. The dyes may also function based on FRET, and therefore may also be comprised of an excitation dye and a reporter dye. Short complementary segments at the 5′ and 3′ terminal regions allow for formation of a stem-loop structure, which positions the dyes at the terminal ends of the oligonucleotide into close proximity, thus resulting in fluorescence quenching or FRET. When the oligonucleotide hybridizes to a PCR product through complementary sequences in the internal region of the molecular beacon probe, fluorescence of the oligonucleotide probe is affected, thus allowing monitoring of product synthesis.

Finally, qPCR may also use fluorescent dyes that preferentially bind to double stranded nucleic acid amplification products during the PCR reaction, thereby providing continuous monitoring of product synthesis (see, e.g., Higuchi, R. et al., Biotechnology 11: 1026-1030 (1993); Morrison, T. B. et al., Biotechniques 24: 954-962 (1998)). Suitable fluorescent dyes include, among others, ethidium bromide, YO PRO-1™ (Ishiguro, T. Anal Biochem. 229: 207-213 (1995)), and SYBR™ Green dyes (Molecular Probes, Eugene, Oreg., USA). Thus in one embodiment of the present invention, qPCR is based on the use of double stranded DNA binding dyes. In one preferred embodiment, the DNA binding dye is SYBR green dye.

Instrumentation suitable for real time monitoring of PCR reactions are available for use in qPCR methods these might include Qiagen's RotorGene Q Qiagen's RotorDisc 100(ABI Prism 7700, Applied Biosystems Division, Perkin Elmer, Fosters City, Calif., USA; LightCycler™, Roche Molecular Biochemicals, Indianapolis, Ind., USA).

The copy number of target nucleic acids may also be determined by comparative quantitative real time PCR. Use of nucleic acids of known copy number or consistent copy number allows quantitating the copy number of target nucleic acids in a sample. The standard may be a single copy gene, a nucleic acid of known copy number, or when quantitating RNA copy number, a constitutively expressed housekeeping gene (see Johnson, M. R. Anal. Biochem. 278: 175-184 (2000); Boulay, J.-L., et al., Biotechniques 27: 228-232 (1999)). Thus in one embodiment the single copy gene is 36b4, β-tubulin, albumin, or other housekeeping genes. In another preferred embodiment, the primers used for the amplification of the 36b4 gene comprise the forward and reverse primers as set forth in SEQ ID Nos.: 3 and 4, respectively. The amplification reactions conditions used for the standard is substantially the same as set forth for the target telomere sequence set forth above. Thus, in one embodiment, the reaction conditions may include a 95° C. enzyme activation step, a 93° C. to 98° C. denaturation step for 5 to 10 seconds and a 58° C. to 60° C. annealing step for 10 to 30 seconds. In a more preferred embodiment, the denaturation step is 98° C. for a duration of 7 seconds and the annealing step is 60° C. for a duration of 7 seconds. The number of cycles may vary between 25 and 35 cycles. In a preferred embodiment, the number of cycles is 35 cycles. It is also an embodiment of the invention that the reaction mixture does not differ from the target telomere gDNA reaction mixture (e.g., the buffers, salts, and enzyme units and primer concentrations are the same or equivalent).

D. Methods of Use

The present invention finds applications in characterizing the functional state of cells, especially for cell changes associated with disease states. Of more defined importance in cell function are tandemly repeating sequences comprising the telomeres of linear eukaryotic chromosomes. The telomeric regions of different organisms differ in their repetitive unit or repeat sequence.

The telomere repetitive unit not only varies between species as to the repeat sequence, but also as to number of repetitive units in an organism. It is well established that the length and integrity of telomeres is important for cell growth and proper segregation of chromosomes.

Thus, measuring the number of repetitive units of specific repetitive sequences find important applications, including, but not limited to, cancer diagnosis, diagnosis of aging related diseases, integrity of cloned organisms, screening of inherited disorders, and drug screening for agents directed to enzymes (i.e., telomerase) and cellular pathways regulating length of repetitive sequences.

Thus, in a preferred embodiment, the present invention provides for rapid analysis of telomere lengths by direct amplification of the repeat sequences using primers capable of primer extension when hybridized to telomere repetitive units. This invention provides for a rapid, accurate, and simple method of determining telomere length. Human telomeric sequences are used herein to illustrate practice of the present invention for direct amplification and quantitation of tandemly repeated nucleic acid sequences, but is not limited to the specific embodiment described herein.

Measuring the number of repetitive units of telomeres has a wide variety of applications in medical diagnosis, disease prognosis, and therapeutics. The present invention is useful for determining telomere lengths of various types of cancer cells since activation of telomerase activity is associated with immortalization of cells. Cells can be analyzed over time to determine whether an increase, decrease, or stabilization of telomeres is associated with disease progression. Various cancer cell types amenable for testing include breast, liver, brain, bone, prostate, lymphocyte, melanoma, colon cancers, etc.

The present invention also finds use in diagnosis of diseases related to early onset of aging. For example, individuals with Hutchinson Gilford progeria disease show premature aging and reduction in proliferative potential in fibroblasts associated with loss of telomeric length (Alssopp, R. C. et al, Proc. Natl. Acad. Sci. USA 89: 10114-10118 (1992)) while patients with dyskeratosis congenita display progressive bone-marrow failure, abnormal skin pigmentation, leukoplakia, and nail dystrophy because of a deletion of telomerase RNA (see Vulliamy, T. Nature 413: 432-435 (2001)). Thus, amplification and quantitation of the number of telomeric repeats is useful for determining the association of particular diseases with changes in telomere length.

In another preferred embodiment, the present invention is useful in monitoring effectiveness of therapeutics or in screening for drug candidates affecting telomere length or telomerase activity. For example, the present invention finds use in monitoring the effectiveness of cancer therapy since the proliferative potential of cells may be related to the maintenance of telomere integrity. The ability to monitor telomere characteristics can provide a window for examining the effectiveness of particular therapies and pharmacological agents. In another aspect, the present invention finds use as a general method of screening for candidate drugs affecting biological pathways regulating telomere length, such as telomerase activity. Ability to rapidly amplify telomere repeats provides a high thorough put screening method for identifying small molecules, candidate nucleic acids, and peptides agents affecting telomere characteristics in the cell.

E. Kits

Also envisioned as one embodiment of the present invention is a kit comprising all of the necessary components and instruments for use the aforementioned methods. Such kits would include all of the premixed reaction mixture (i.e., buffers, salts, additives, etc.), primers, a polymerase, detection tools, and the instruments for amplification. Thus in one embodiments of the invention, a kit would include a primer set, Taq DNA polymerase, a premixed and amplification buffer, salt, and additive mixture, instructions for amplification. In one embodiment, the kit would include a primer set of SEQ ID Nos.: 1 and 2, a HotStar Taq plus DNA polymerase, a premixed reaction mixture, appropriate dyes for detection, and an instruction pamphlet provided for optimized reaction conditions. In another embodiment, the kit may further comprise a set of single gene reference primers comprising SEQ ID Nos.: 3 and 4. In another embodiment, the kit may also include a thermocycler for the use in the above described kit.

### EXAMPLES

The qPCR method of the present invention is highly accurate, reproducible, and simple for human peripheral cells. The sensitivity of the reaction is enhanced, requiring limited reaction volumes of 20-μl and target telomere DNA amounts of 1 ng/assay. These assays provided correlation of R²=0.9939, slope=−3.13 and amplification efficiency at 100%. Further, the qPCR assay takes only 47 min for 100 telomere targets.

### A. Example 1

To determine if the amplification of a telomere hexameric repeat sequence would work using commercially available qPCR kits and qPCR instruments, an amplification reaction was performed using QIAGEN chemistry on a RotorGene cycler. Telomere gDNA was amplified using standard reaction conditions of 95° C. for 5 minutes, 95° C. for 5 seconds, 58° C. for 20 seconds for 40 cycles as outlined by Cawthon in the 2002 (see Cawthon pg. e47-52). Table 1 demonstrates that the amplification of the telomere and single copy reference gene (36b4) gDNA was possible using the standard instrumentation and reaction conditions.

TABLE 1

| Sample | Rep. Ct | Rep. Ct Std. Dev. | Tm |
|---|---|---|---|
| Telo_09_20ng | 12.24 | 0.05 | 83 |
| Telo_17_20ng | 12.8 | 0.12 | 83 |
| Telo_25_20ng | 11.48 | 0.02 | 83 |
| Telo_26_20ng | 11.88 | 0.02 | 83 |
| Telo_1266_20ng | 12.79 | 0.08 | 83 |
| Telo_NTC | 25.95 | 0.15 | 81 |
| 36b4_09_20ng | 23.43 | 0.05 | 79.5 |
| 36b4_17_20ng | 23.62 | 0.05 | 79.5 |
| 36b4_25_20ng | 23.22 | 0.04 | 79.5 |
| 36b4_26_20ng | 23.18 | 0.03 | 79.5 |
| 36b4_1266_20ng | 23.11 | 0.04 | 79.5 |
| 36b4_NTC | n/a | n/a | n/a |

### B. Example 2

A standard curve was used to assess an adequate concentration of starting telomere genomic DNA that could be used in the assay. A qPCR amplification reaction consisting of 25-cycles (running for a total time of 47 minutes) was performed on Rotor-Gene® Q real-time instrument with QIAGEN Rotor-Gene® SYBR Green Kit. Human sample are diluted at 1:2 in a seven series dilution, resulting in a range of 0.1 ng to 6.25 ng/PCR in a 20-μl reaction volume. The PCR condition used in the assay was 95° C. 5 s; 98° C. 7 s, 60° C. 10 s (25 cycles). The assay was set up by QIAgility liquid handling instrument. The primers used in this amplification reaction were SEQ ID Nos.: 1 and 2.

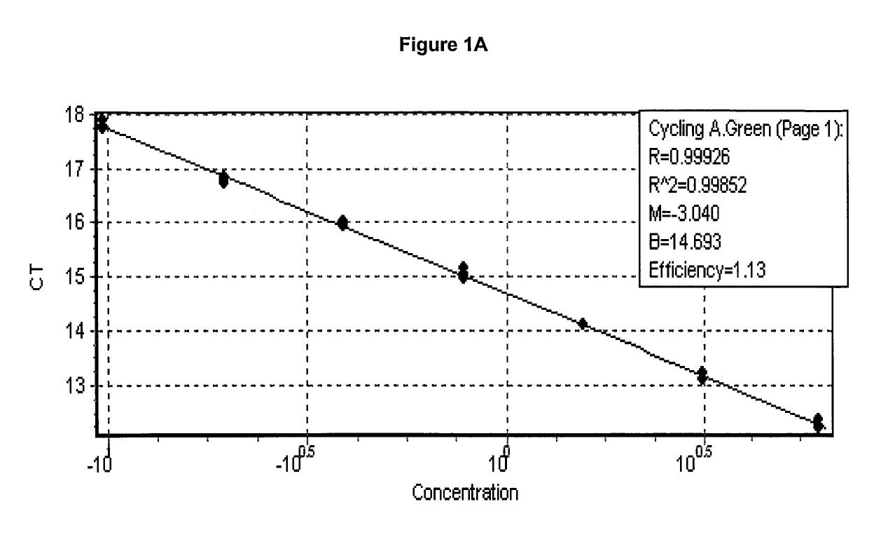

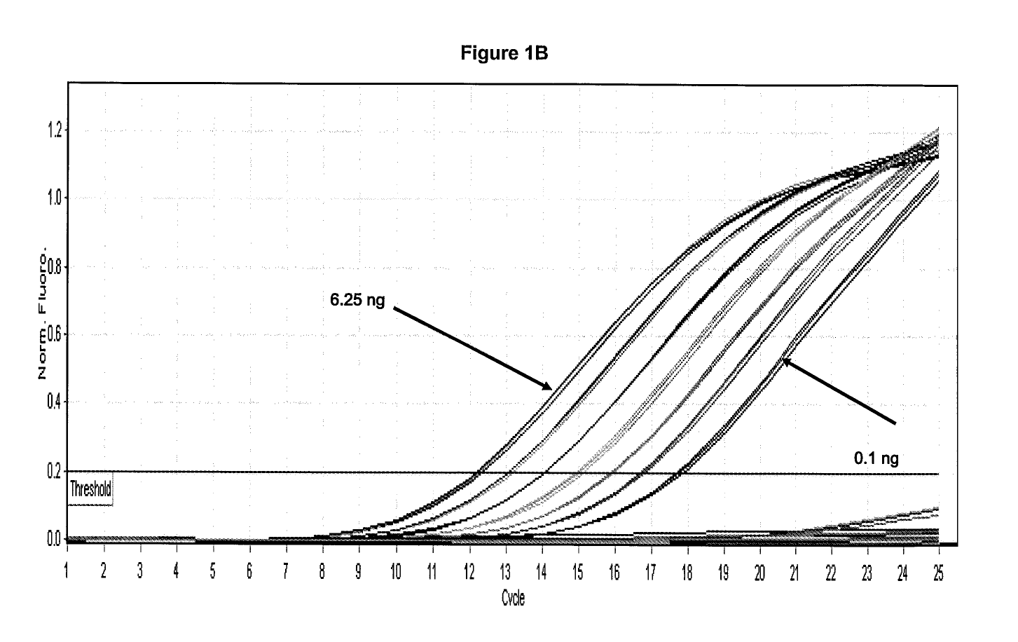

FIG. 1 demonstrates and establishes a standard curve for the concentration of telomere gDNA in the range of 0.1 ng to 6.25 ng. The amplification using the lower concentrations of telomere gDNA produced a slope=−3.04, R²=0.99852, with an enzyme efficiency of 1.13.

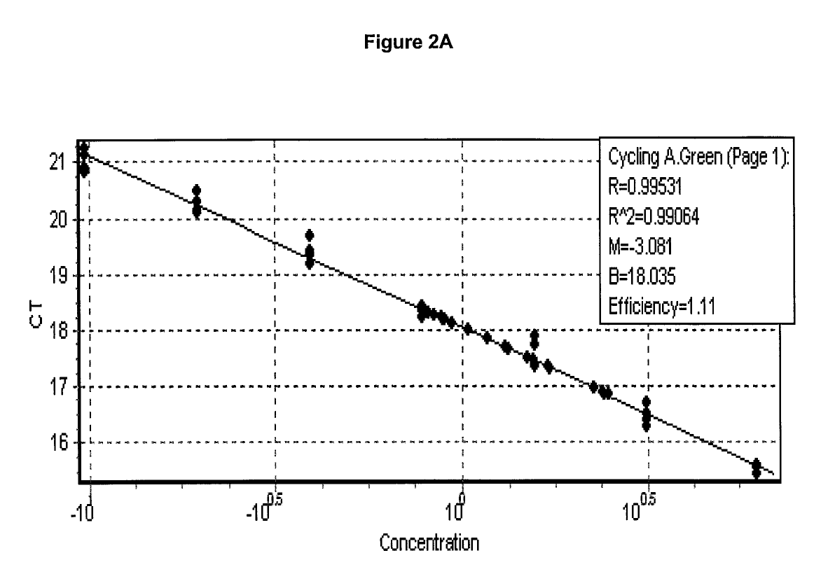

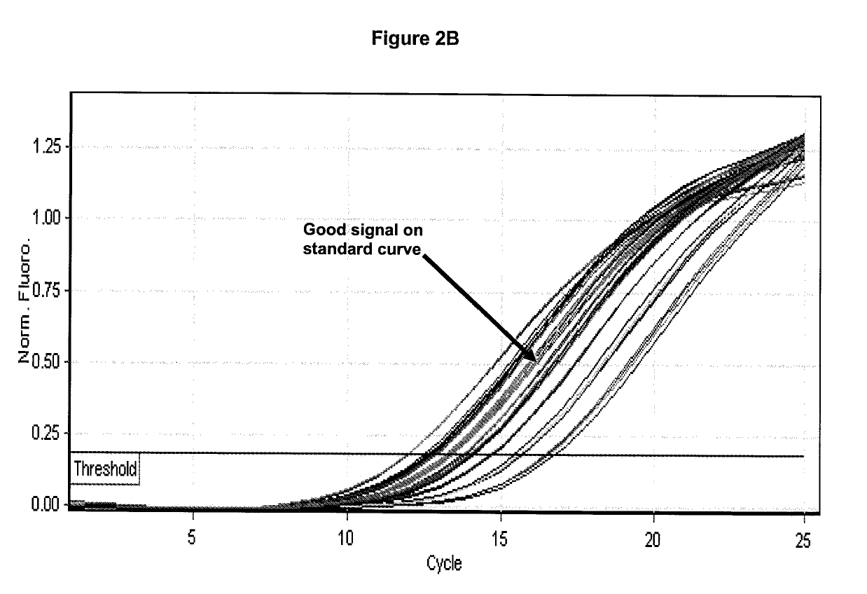

Using QIAGEN Rotor-Disc 100, the telomere assay was conducted using the previously determined lowered concentrations of telomere gDNA. Multiple human samples of 1 ng/PCR reaction were used along with a standard curve of seven point serial dilutions in the range of 0.1 ng to 6.25 ng/PCR reaction. Using the same reaction conditions of 95° C. 5 s; 98° C. 7 s, 60° C. 10 s (25 cycles), it was determined that 1 ng per PCR reaction was optimal. As demonstrated in FIG. 2, the amplification signal, corresponding to 1 ng of telomere gDNA, falls within the middle of the standard curve indicating that it provided an optimal concentration for the assay. This assay provided a slope of −3.081, a R² of 0.99064, and an enzyme efficiency of 1.11.

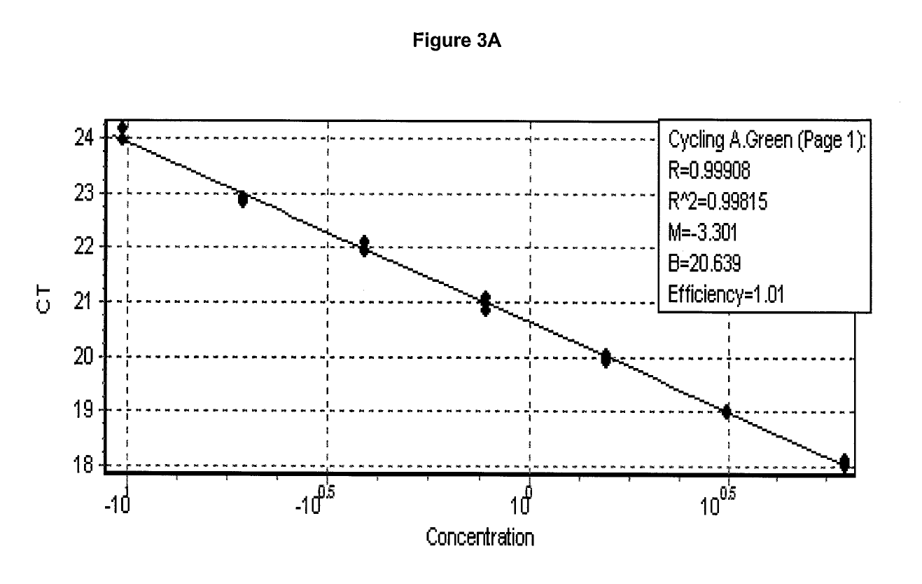

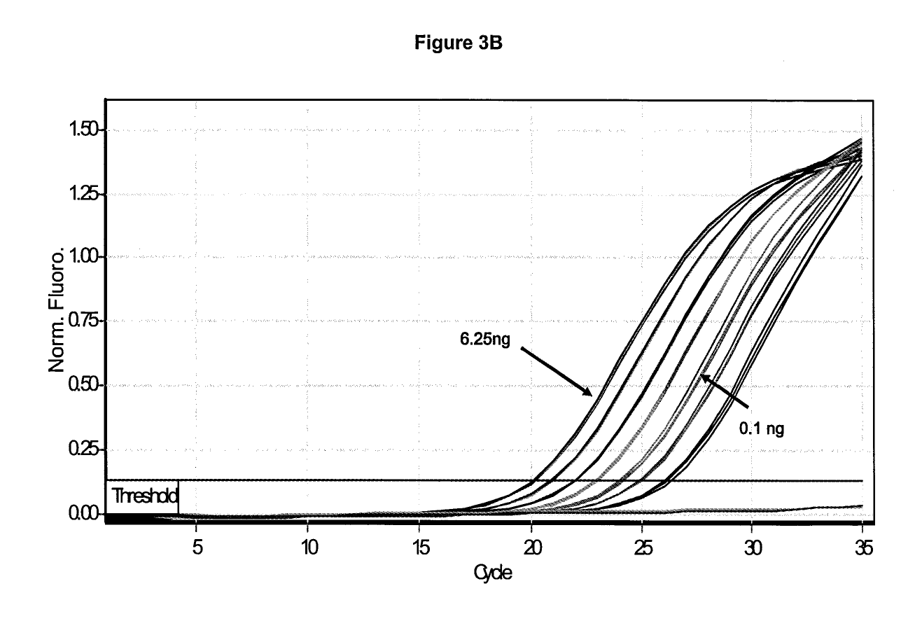

A comparative assay using a single copy gene as a reference is performed to determine whether the reaction conditions were acceptable. Accordingly, a 35-cycle, 61 minute qPCR reaction is performed on the Rotor-Gene® Q real-time instrument with QIAGEN Rotor-Gene® SYBR Green Kit. Single copy gene gDNA (36b4) from human sample is diluted at 1:2 in seven series dilutions in the range of 0.1 ng-6.25 ng/PCR reaction in a 20-μl reaction volume. The PCR condition used in the assay was 95° C. 5 s; 98° C. 7 s, 58° C. 10 s (35 cycles). The assay was set up by QIAgility liquid handling instrument. Primers used in this assay were SEQ ID Nos.: 3 and 4. FIG. 3 demonstrates and establishes a standard curve utilizing a range of 0.1 ng to 6.25 ng/PCR reaction. The amplification using the lower concentrations of single copy gene reference 35b4 gDNA produced a slope=−3.301, R²=0.99815, with an enzyme efficiency of 1.01.

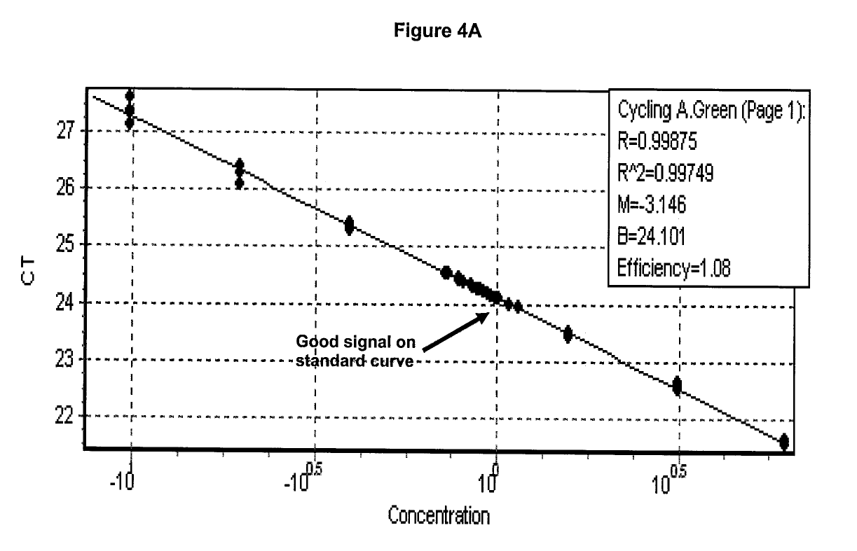

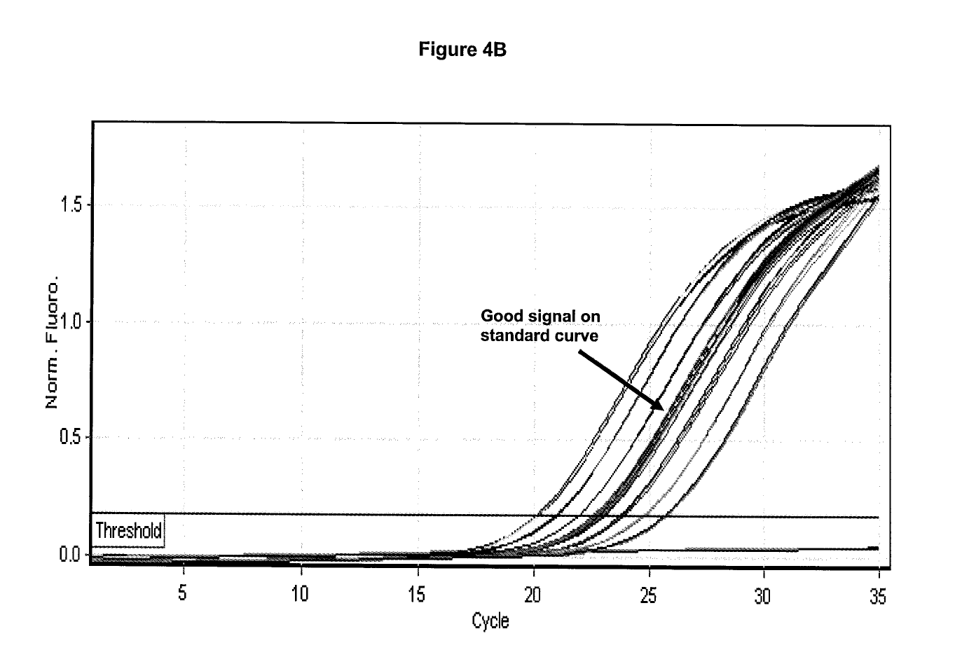

The assay was repeated using the gDNA concentrations determined in FIG. 3 using the same reaction conditions of 35-cycle, 61 min PCR at 95° C. 5 s; 98° C. 7 s, 58° C. 10 s on Rotor-Gene® Q real-time instrument with QIAGEN Rotor-Gene® SYBR Green Kit on a 100-Rotor-Disc™ ring. Multiple human samples at 2 ng/PCR reaction is used along with a seven series dilutions curve (in the range of 0.1 ng-6.25 ng/PCR) in a 20-μl reaction volume set up by QIAgility, an automated liquid-handling instrument. FIG. 4 demonstrates that the concentration of 1 ng/PCR reaction of human 36b4 gDNA falls in the middle of the standard curve. This assay provided a slope of −3.146, a R² of 0.99749, and an enzyme efficiency of 1.08.

### C. Example 3

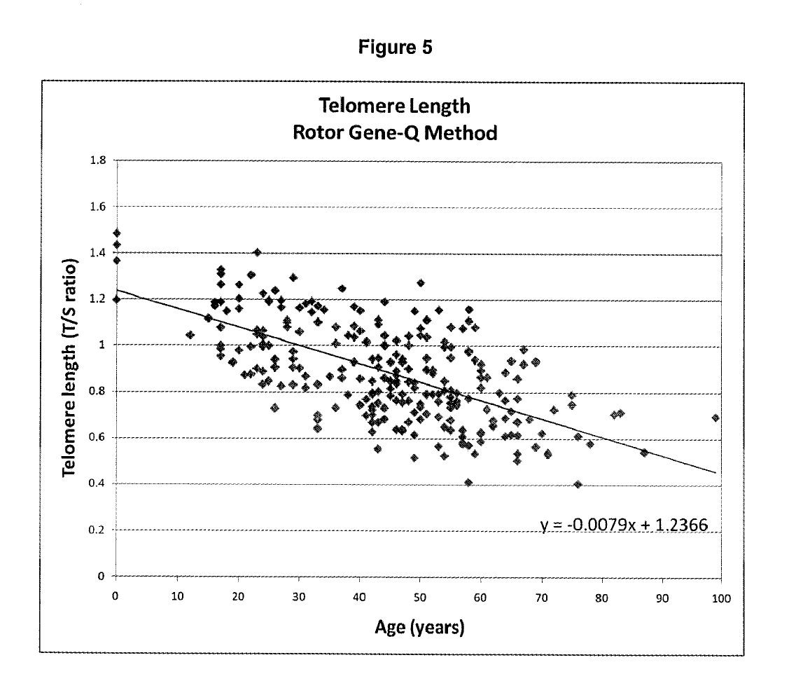

The telomere length was measured in 13 samples using the gold standard Southern blot method and the new qPCR method described herein. Cₜ values form the telomere assay were normalized to the single gene reference assay using the T/S ratio to determine telomere length. The telomere length (T/S ratio) from real time PCR was then correlated against telomere length as determined by Southern blotting analysis. As shown in FIG. 5, the telomere length (x) from each sample was based on the telomere to single gene copy ratio (T/S ratio) and was based on the calculation of the ΔCₜ [Cₜ^{(telomere)}/Cₜ^{(single gene)}]. Telomere length is expressed as a relative T/S ratio, which was normalized to the average T/S ratio of the reference sample [2−^{(ΔCtx-ΔCtr)}=2^{-ΔΔCt}] for all standard curves, reference samples, and validation samples. In order to make results comparable from different assay runs, the results of each run is approved only if the relative T/S ratio of the validation reference sample falls within a 3% variation. This demonstrated that there is a high correlation between the two methods (R²=0.8623), significantly higher than the originally described (Cawthon. NAS 2002).

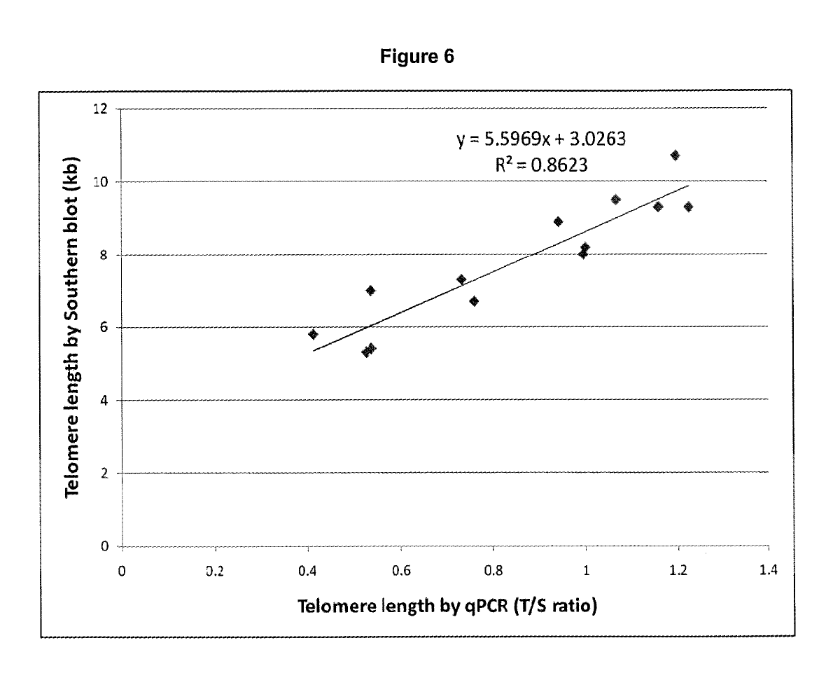

An assay with 299 healthy human subjects to date spread out in each age group ranging from 0 (core blood) to 99-year of age is performed. The results are positive with an inverse correlation between telomere length and age. The telomere length was measured in 13 samples using the gold standard Southern Blot method and the new quantitative PCR method. FIG. 6 demonstrates that there is a higher degree of correlation (R²=0.8623), which is significantly higher than originally described 67% (R²=0.6771) by Cawthon (NAS 2002). Table 2 summaries these results.

TABLE 2

| Description | q-PCR | | Southern Blot | |
|---|---|---|---|---|
| No. samples tested | 299 | | 13 | |
| Total time (Assay; Analysis) | 47 | min | 2 | days |
| Correlation; R² | 0.9939 | | 0.8623 | |
| Sensitivity: Sample (gDNA) required | 1 | ng | 200 | ng |

This method has potential applications in the clinical use for the diagnosis of age related telomere diseases and conditions such as: Macular degeneration (vision loss), Atherosclerosis (hardening of arteries by plaques), Impaired wound healing, Heart disease, Gray hair, Wrinkles.

We believe this fast, sensitive methodology is a powerful tool to study genomic instability, heart attack and strokes, cancer progression and therapy. It can provides new avenue for investigators to address the full extent and significance of telomere shortening in the carcinogenic process. It allows for more widespread use of this technique among cancer researchers and for study of age related telomere diseases and conditions.

The compositions and methods described above find use in any process for amplifying target nucleic acids by polymerase chain reaction. Thus, the present invention is useful in detecting and monitoring infectious diseases, for example in testing for presence of pathogenic bacteria and viruses (e.g., viral load). For instance, target viral nucleic acids include, without limitation, HIV, HCV cytomegalovirus, hepatitis, etc. The present invention is also applicable for monitoring medical therapies. For example this may involve monitoring the progress of bacterial infections following antibiotic administration.

All references cited herein are incorporated by reference in their entireties.

1. A method of measuring a repeating nucleotide sequence in a telomere region comprising the steps of:
(a) extracting and purifying at least one DNA sample or target DNA template from a subject specimen;
(b) amplifying the DNA sample in a reaction mixture at high denaturing and annealing temperatures; and
(c) detecting the amplified DNA in the presence of a labeled probe,
wherein the entire process is completed in under one hour.

2. The method of claim 1, wherein the measuring is to diagnose or detect the presence of a disease or condition selected from age related telomere disease or condition is selected from bone marrow failure, leukemia, macular degeneration, atherosclerosis, impaired wound healing, heart disease, wrinkling, or age related graying of hair.

3. The method of claim 1, wherein the repeating nucleotide sequence comprises 5′-TTAGGG-3′ as shown in SEQ. ID. No.: 1.

4. The method of claim 1, wherein the DNA is extracted and purified from tissues, peripheral cells, or peripheral blood cells.

5. The method of claim 1, wherein the amplification is a polymerase chain reaction.

6. The method of claim 5, wherein the polymerase chain reaction is a quantitative real time polymerase chain reaction.

7. The method of claim 1, wherein the reagents comprise at least a first set of primers that hybridize under stringent conditions to a region that detects the presence of a nucleotide sequence of SEQ. ID. No.: 1.

8. The method of claim 7, wherein the first set of at least two primers is completely complementary to the DNA sample or target template DNA.

9. The method of claim 8, wherein the first set of at least two primers comprises a forward primer as shown in SEQ ID No.: 1 and a reverse primer as shown in SEQ ID No.: 2.

10. The method of claim 1, wherein the reagents comprise an agent capable of changing the melting behavior of double stranded nucleic acid molecules.

11. The method of claim 10, wherein the agent is selected from betaine.

12. The method of claim 10, wherein the betaine is solution Q.

13. The method of claim 1, wherein the reagents comprise a synthetic factor capable of enhancing multiplex and/or primer annealing.

14. The method of claim 13, wherein the synthetic factor is Factor MP.

15. The method of claim 1, wherein the high denaturing temperature is in the range of about 93° C. to about 98° C.

16. The method of claim 15, wherein the high denaturing temperature is 98° C.

17. The method of claim 15, wherein the denaturing temperature is held at constant for a duration of about 5 seconds to about 15 seconds.

18. The method of claim 17, wherein the denaturing temperature is held constant for a duration of 10 seconds.

19. The method of claim 1, wherein the annealing temperature is in the range of about 58° C. to about 60° C.

20. The method of claim 1, wherein the annealing temperature is 60° C.

21. The method of claim 1, wherein the annealing temperature is held constant for a duration of about 5 seconds to about 30 seconds.

22. The methods of claim 21, wherein the annealing temperature is held constant for a duration of about 10 seconds.

23. The method of claim 5, wherein the amplification is compared to an internal control.

24. The method of claim 23, wherein the internal control is a single copy gene.

25. The method of claim 24, wherein the single copy gene is 36b4.

26. The method of claim 25, wherein the single copy gene 36b4 is amplified using two primers comprising a forward primer as shown in SEQ ID No.: 3 and a reverse primer as shown in SEQ ID No.: 4.

27. The method of claim 1, wherein the reagents further comprise a detectable label.

28. The method of claim 27, wherein the detectable label are fluorescent dyes.

29. The method of claim 27, wherein the detectable label specifically binds to double stranded nucleic acids.

30. The method of claim 27, wherein the detectable label is selected from SYBR® Green I, SYBR® Gold, ethidium bromide, propidium bromide, Pico Green, Hoechst 33258, YO-PRO-I and YO-YO-I, Boxto, Evagreen, LC Green, LC Green Plus and Syto 9.

31. The method of claim 27, wherein the detectable label is SYBR® Green I.

32. The method of claim 1, wherein the amplification is repeated for 25 cycles to 35 cycles.

33. The method of claim 32, wherein the period is 35 cycles.

34. The method of claim 32, wherein the period of 25 cycles.

35. The method of claim 1, wherein the DNA sample is in an amount of about 0.1 ng to about 20 ng.

36. The method of claim 35, wherein the DNA sample is in an amount of about 0.1 ng to 10 ng.

37. The method of claim 35, wherein the DNA sample is in an amount of about 0.1 ng to 6.25 ng.

38. The method of claim 1, wherein the DNA is genomic DNA.


SEQUENCE LISTING (4 sequences)

SEQ ID NO: 1 - DNA, 39 bp, Human
cggtttgtttgggtttgggtttgggtttgggtttgggtt

SEQ ID NO: 2 - DNA, 39 bp, Human
ggcttgccttacccttacccttacccttacccttaccct

SEQ ID NO: 3 - DNA, 23 bp, Human
cagcaagtgggaaggtgtaatcc

SEQ ID NO: 4 - DNA, 25 bp, Human
cccattctatcatcaacgggtacaa